(19) United States
(12) Patent Application Publication
(10) Pub. No.: US 2011/0177090 A1
(43) Pub. Date: Jul. 21, 2011

(54) Glycan markers and autoantibody signatures in HIV-1 and HIV-1-associated malignancies

(75) Inventors: Denong Wang, Palo Alto, CA (US); Leonore A. Herzenberg, Stanford, CA (US)

(73) Assignee: The Board of Trustees of the Leland Stanford Junior University, Palo Alto, CA (US)

(21) Appl. No.: 13/009,474

(22) Filed: Jan. 19, 2011

Related U.S. Application Data
(60) Provisional application No. 61/296,317, filed on Jan. 19, 2010.

(51) Int. Cl. A61K 39/42; C40B 40/12; C40B 30/04; C07K 16/10; C40B 40/10; A61P 31/18; A61P 37/04

(52) U.S. Cl. 424/148.1; 506/19; 506/9; 530/388.35; 530/388.15; 530/387.3; 506/18

(57) ABSTRACT

The present invention provides compositions and methods for the specific and sensitive detection and characterization of anti-carbohydrate antibodies against immunogenic sugar moieties on microbes and abnormal cells and anti-HIV-1 protein antibodies for the development of diagnostic, prophylactic and therapeutic strategies against diseases inflicted by cancer cells or microbial pathogens.

### RELATED APPLICATION

This application claims priority and other benefits from U.S. Provisional Patent Application Ser. No. 61/296,317, filed Jan. 19, 2010, entitled “Glycan markers and autoantibody signatures in HIV-1 and HIV-1-associated malignancies”. Its entire content is specifically incorporated herein by reference.

### STATEMENT OF GOVERNMENTAL SUPPORT

This invention was made with U.S. Government support under UO1 CA128416, UO1 CA128416-52 and R21AI064104 awarded by the National Institutes of Health. The Government has certain rights in this invention.

### TECHNICAL FIELD OF THE INVENTION

The present invention relates to the field of glycan biomarkers and anti-glycan antibodies against HIV-1 and other antigens for the detection of HIV-1 and HIV-1-associated malignancies and for diagnostic, prognostic and therapeutic intervention strategies including vaccination against HIV-1 and HIV-1-associated malignancies.

### BACKGROUND

Sugar moieties are abundantly expressed on the outer cell surface of many microbes and host cells. In bacterial, protozoan and fungal pathogens, many sugar structures are highly specific for a given pathogen, which makes them important molecular targets for pathogen recognition, diagnosis of infectious diseases, and vaccine development. Sugar moieties are also expressed and displayed on the surfaces of virions in almost all known viral species infecting mammals.

Viruses take advantage of the cellular machineries of carbohydrate synthesis and protein glycosylation to produce viral carbohydrates and glycoproteins. A prominent example is the glycosylation of the gp120 glycoproteins of different HIV-1 strains with high-mannose type N-glycans. Glycosylation products can contain immunogenic carbohydrate moieties that could be immunological targets for the development of prophylactic and therapeutic strategies against various diseases. However, the induction and identification of antibodies against those viral carbohydrate moieties has remained a challenge, partly due to their structural similarities with self-carbohydrates and partly due to technical challenges and lack of sensitive and specific detection.

Need for Methodologies and Assays

The exploration of immunogenic sugar moieties that are important for “self” and “non-self” discrimination and host immune responses is an important focus in the continuing search for effective, carbohydrate-based prophylactic and therapeutic immunotherapy strategies against various diseases. However, technical challenges to identification, characterization and highly sensitive as well as specific detection are substantial and have hampered the development of prophylactic and therapeutic strategies against diseases inflicted by abnormal cells or microbial cells.

### SUMMARY

Addressing and overcoming the above described difficulties, the described invention provides methods and compositions for the specific and sensitive detection and characterization of anti-carbohydrate antibodies against immunogenic sugar moieties on microbes and abnormal cells and for the development of diagnostic, prophylactic and therapeutic strategies against diseases inflicted by cancer cells or microbial pathogens.

In one particular embodiment, the invention provides a carbohydrate cluster microarray platform for detecting 2G12-like anti-high mannose cluster antibodies in HIV-1 infected and AIDS-afflicted human subjects.

In another embodiment, the invention provides a carbohydrate cluster microarray platform for diagnosis and prognosis of AIDS-associated malignancies.

In another embodiment, the invention provides carbohydrate cluster microarray-based methods for detecting 2G12-like anti-high mannose cluster monoclonal antibodies, lectins and other receptors.

In a further embodiment, the invention provides antibodies for defining 2G12-like novel cryptic glyco-epitopes displayed by high-mannose clusters.

In other embodiments, the invention provides methods for inducing anti-Man9-cluster immunity against HIV-1 infection.

In more embodiments, the invention provides methods for inducing anti-Man9-cluster immunity for treatment of HIV-1 infection, alone or in combination with other anti-viral therapies.

In further embodiments, the invention provides methods for inducing anti-Man9-cluster immunity against HIV-1-associated malignancies. In other embodiments, the invention provides methods for inducing anti-Man9-cluster immunity for treatment of HIV-1-associated malignancies, alone or in combination with other anti-viral therapies and/or anti-cancer drugs.

The above summary is not intended to include all features and aspects of the present invention nor does it imply that the invention must include all features and aspects discussed in this summary.

### INCORPORATION BY REFERENCE

All publications and patent applications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1A displays a carbohydrate microarray analysis to examine the binding of the monoclonal antibodies G1(PrCa-X) and AE3 against the human carcinoma antigen (HCA). Forty eight* glycoproteins and neoglycoconjugates were spotted in triplicates and in two dilutions to yield a total of 288 microspots per microarray slide. Images of microarrays stained with (A) lectin Helix pomatia agglutinin (HPA), which is highly cross-reactive with Gal/GalNAc-terminated glyco-epitopes and serves as a reagent for monitoring efficacy of immobilization of Gal-containing glycoconjugates; (B) Anti-mouse IgM alone; (C) mAb AE3 and (D) G1(PrCa-X). *Only 12 of the glycoconjugates tested are shown; the rest are negative as statistically measured with cut-off of˜ratio 1.5, illustrating the specificities of G1(PrCa-X)/AE3.

FIG. 1B shows the glycan binding profiles of three anti-Man9 mAbs and their cross-reactivities with HIV-1 gp120 glycoproteins. 2G12, G1(PrCa-X) and TM10 were applied on glycan arrays at 10 μg/ml, 5 μg/ml and 5 μg/ml, respectively. G1(PrCa-X) and TM10 were obtained by cancer immunizations and are highly and selectively reactive with a number of human cancers. Importantly, G1(PrCa-X) is highly reactive with metastatic prostate carcinoma in bone.

FIG. 2 illustrates high mannose (Man9)-clusters (mannose, ▪GlcNAc) in different structural configurations bound to a carrier, for example, keyhole limpet hemocyanin (KLH) or bovine serum albumin (BSA), through a linker (R1) and a thiol (S). The linker can be Asn-Ac-SH, the bivalent maleimide cluster MC-2, the trivalent maleimide cluster MC-3, or the galactose-based maleimide cluster MC-1 (Wang et al., 2004).

FIG. 3 shows that HIV-1 infection elicits anti-Man9 antibodies in human subjects.

FIG. 4 illustrates a comparison of relative antibody reactivities among five carbohydrate antigens, including four mannose-containing antigens and one alpha-Gal antigen.

FIG. 5 shows in SJL/J mice, upon a single injection of a CFA emulsion containing Man9-KLH and myelin proteolipid protein (PLP), and induction of anti-Man9-cluster IgG antibodies that are highly cross-reactive with HIV-1 gp120 glycoproteins.

FIG. 6 illustrates antibody responses to HIV-1 gp120 glycoproteins upon immunization with Man9-KLH. Co-immunization of SJL mice with Man9-KLH (100 μg/mouse) and PLP₁₃₉₋₁₅₁ (100 μg/mouse) emulsified in CFA.

### DEFINITIONS

The practice of the present invention may employ conventional techniques of chemistry, molecular biology, recombinant DNA, genetics, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique', Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The terms “glyco-epitope” and “carbohydrate epitope” are used interchangeably and characterize a region of an oligosaccharide or polysaccharide chain exhibiting a multitude of sugar residues on the surface of an antigen that is specifically or selectively recognized by an antibody, a lectin and/or other receptors. Glyco-epitopes can be formed by oligosaccharides and can include monosaccharides such as mannose (man), galactose (gal) and glucose (glc).

The term “polysaccharide” refers to a polymeric sugar or so-called ‘glycan’, consisting of a plurality of monosaccharide residues that are joined with each other through glycosidic linkages. Polysaccharides with residues of less than 10 members are usually termed ‘oligosaccharides’ in the art.

The term “N-glycan” refers to an N-linked oligomeric or polymeric sugar chain that is attached to a protein or lipid through an asparagine-N-acetyl-D-glucosamine linkage. N-glycans can have different numbers of branches comprising various monosaccharides that are attached to the core structure. N-acetylglucosamine is typically referred to as GlcNAc.

The terms “mannose core”, “mannose core structure” or “core structure”, as used herein, refer to oligo- or polysaccharides which exhibit different sugar branches and which terminate in a number of mannose monosaccharide units. Man9 denominates nine units of mannose per branch attached to two GlcNAc residues, so, for example, [(Man9)4] describes a mannose core structure with four branches of mannose units, whereby each branch consists of nine units of mannose. Accordingly, Man8 denominates eight units of mannose per branch attached to two GlcNAc residues, Man7 denominates seven units of mannose per branch attached to two GlcNAc residues, Man6 denominates six units of mannose per branch attached to two GlcNAc residues and Man5 denominates five units of mannose per branch attached to two GlcNAc residues. Prominent mannose core structures herein are (Man9)ₙ which represents the mannose-core of N-glycoproteins and [(Man9)4]ₙ which mimics the mannose clusters displayed by the gp120 glycoprotein of HIV-1; n indicates multiple repeats of a mannose monosaccharide unit, indicating that a random number of units can be attached to a single carrier. Both mannose (Man9)ₙ and [(Man9)4]ₙ clusters were preferably bound to the carrier keyhole limpet hemocyanin (KLH), which supports the Man9 clustering, but other carriers, such as a bovine serum albumin (BSA) carrier, may be used as well. High-mannose chains are defined as mannose clusters with mannose core structures ranging from Man5 to Man9.

The term “biological sample” encompasses any sample consisting of or containing blood, serum, plasma, lymph fluid, amniotic fluid, saliva, cerebro-spinal fluid, lacrimal fluid, mucus, urine, sputum, or sweat.

The term “antibody” relates to antibodies of all possible types, in particular to monoclonal antibodies and also to antibodies produced by chemical, biochemical or genetic technological methods. The term “antibody” further includes various forms of modified or altered antibodies, such as various derivatives or fragments such as an Fv fragment, an Fv fragment containing only the light and heavy chain variable regions, an Fv fragment linked by a disulfide bond, a Fab or (Fab)'2 fragment containing the variable regions and parts of the constant regions, a single-chain antibody and the like. The antibody may be of animal (especially mouse or rat) or human origin or may be chimeric. It may be humanized.

The term “carrier protein”, as used herein, means a protein suitable for conjugation to a high-mannose cluster including, but not limited to keyhole limpet hemocyanin (KLH), tetanus toxoid, diphtheria toxoid, bovine serum albumin and/or ovalbumin.

The term “vaccine”, as used herein, means a biological or pharmaceutical preparation or product that improves immunity to a particular disease. A ‘therapeutic’ vaccine is a vaccine that helps fight a particular disease that already has set in, while a ‘prophylactic’ vaccine is a vaccine that is meant to prevent a particular disease.

The term “passive vaccination” means evoking a specific, “passive” immunity due to administration of antibodies against a glyco-epitope or carbohydrate epitope. The immunity is “passive”, because the organism does not actually create antibodies itself, but only utilizes the administered antibodies. The passive immunity is usually short-term, lasting from several days to several months. Passive immunity can be transferred through the administration of serum that contains particular antibodies. A vaccination can, in principle, be either carried out in a prophylactic or therapeutic fashion. A “prophylactic” vaccination is a vaccination administered to a human subject prior to detection of any disease such as HIV-1-infection. A “therapeutic” vaccination is a vaccination administered to a human subject in whom a disease such as HIV-1 has been diagnosed.

“Active” immunity means that the organism itself produces antibodies through the activation of B-cells and T-cells either as a consequence of an exposure to a pathogen or as a consequence of the exposure to an antigen in the context of a vaccination.

“Immunity”, as used herein, means a specific host immune response that provides sufficient biological defenses to fight off a disease or pathological state temporarily or permanently.

The term “normal cell”, as used herein, characterizes a cell that exhibits regular cell division, while “abnormal”, as used herein, indicates unregular, possibly uncontrolled cell division.

The term “immunogenic”, as used herein, relates to the ability of a particular antigen or epitope to provoke an immune response, i.e., a reaction from the immune system within an organism to protect against a disease by identifying and/or killing pathogens and/or abnormal cells.

The term “glycoprotein”, as used herein, relates to proteins that contain oligosaccharide chains (glycans) covalently attached to polypeptide side-chains. In N-glycoproteins, the glycans are attached to the nitrogen of a nitrogen-containing residue, while in O-glycoproteins, the glycans are attached to the oxygen of an oxygen-containing residue.

The term “glycolipid”, as used herein, relates to lipids that have glycans covalently attached.

The term “glycoconjugates”, as used herein, relates generally to carbohydrates that are covalently linked with other chemical species and include glycoproteins and glycolipids.

The term “glyconeoconjugates”, as used herein, relates generally to carbohydrates that are covalently linked with non-naturally occurring molecules.

The term “antigen” refers to any substance that can stimulate the production of antibodies and can combine specifically with them. The term “antigenic determinant” or “epitope”, as used herein, refers to an antigenic site on a molecule.

The term “humanized monoclonal antibodies”, as used herein, refers to antibodies in which the complementarity determining regions (“CDRs”), which exhibit the antibody binding sites of a mouse monoclonal antibody, are replaced with CDRs of the corresponding human protein, while maintaining the framework and constant regions of the mouse antibody.

The term “disease” or “disorder”, as used herein, refers to an impairment of health or a condition of abnormal functioning.

The term “2G12-like”, as used herein, indicates having immunogenic properties that are exhibited by the broadly HIV-1-neutralizing monoclonal antibody 2G12, but that are not identical to 2G12 or the 2G12-epitope.

The term “non 2G12”, as used herein, indicates having immunogenic properties that are not or only to a negligible degree exhibited by the broadly HIV-1-neutralizing monoclonal antibody 2G12, but exhibited by other antibodies, such as G1(PrCa-X) and TM10, which are cross-reactive with HIV-1 glycoproteins.

### DETAILED DESCRIPTION

Embodiments of the present invention provide methods and compositions for the specific and sensitive detection and characterization of anti-carbohydrate antibodies against immunogenic sugar moieties on microbes and abnormal cells and for the development of prophylactic and therapeutic strategies against diseases inflicted by abnormal cells or microbial cells.

### Applicability to HIV-1 and HIV-1-Associated Malignancies

HIV

Human immunodeficiency virus (HIV) is a retrovirus that causes the acquired immunodeficiency syndrome (AIDS), a condition in humans in which the immune system begins to fail, leading to life-threatening opportunistic infections. Retroviruses are viruses that contain their genetic material in form of ribonucleic acid (RNA). After infecting a cell, HIV utilizes the enzyme reverse transcriptase to convert its RNA into deoxyribonucleic acid (DNA) and then proceeds to replicate itself using the host cell's transcription and translation machinery (‘host cell machinery’). Infection with HIV occurs by the transfer of bodily fluids, in particular by the transfer of blood or semen. Within these bodily fluids, HIV is present as both free virus particles and virus within infected immune cells. The four major routes of transmission are unsafe sex, contaminated needles, breast milk, and transmission from an infected mother to her baby at birth (vertical transmission). Screening of blood products for HIV has largely eliminated transmission through blood transfusions or infected blood products in the developed world.

HIV infection in humans is considered pandemic by the World Health Organization (WHO). HIV infects about 0.6% of the world's population. Since its discovery in 1981, AIDS has claimed millions of lives, both adults and children. A third of these deaths are occurring in sub-Saharan Africa, retarding economic growth and increasing poverty. Antiretroviral treatment reduces both the mortality and the morbidity of HIV infection, but routine access to antiretroviral medication is not available in all countries.

HIV infects primarily vital cells in the human immune system such as CD4+ helper cells, macrophages and dendritic cells. CD4+ helper T cells are important in initiating and propagating an initial immune response by activating and directing other immune cells, macrophages' primary task is to phagocytose cellular debris and pathogens, while dendritic cells help to propagate immune response as antigen-presenting cells. Ultimately, HIV-1 infection compromises the immune system through the gradual depletion of CD4+T cells either via direct viral killing of infected cells, through enhanced T cell apoptosis and through killing of infected CD4⁺ T cells by CD8 cytotoxic lymphocytes that recognize infected cells. When CD4⁺ T cell numbers decline below a critical level, cell-mediated immunity is lost, and the body becomes progressively more susceptible to opportunistic infections.

Most people infected with HIV eventually develop AIDS. These individuals mostly die from opportunistic infections or HIV-associated malignancies as a result of the progressive failure of the immune system. HIV progresses to AIDS at a variable rate affected by viral, host, and environmental factors; HIV-specific treatment delays this process. Most will progress to AIDS within 10 years of HIV infection. Treatment with anti-retrovirals increases the life expectancy of people infected with HIV. Even after HIV has progressed to diagnosable AIDS, the average survival time with antiretroviral therapy is estimated to be several years.

There are two species of HIV known to exist: HIV-1 and HIV-2. HIV-1 is the virus that was initially discovered and termed LAV. It is more virulent, more infectious and is the cause of the majority of HIV infections world-wide. The lower infectivity of HIV-2 compared to HIV-1 implies that fewer of those exposed to HIV-2 will be infected per exposure. Because of its relatively poor capacity for transmission, HIV-2 is largely confined to West Africa.

HIV Proteins

The HIV-1 virus belongs to the genus of lentiviruses which are characterized by a long incubation period. Therefore, a person who is infected with HIV-1 might not exhibit symptoms of AIDS for several years. Lentiviruses can deliver a significant amount of genetic information into the DNA of the host cell and have the unique ability among retroviruses to replicate in non-dividing cells, so they are one of the most efficient methods of a gene delivery vector. HIV-1 does not exclusively rely on the host cell machinery, but also produces a number of viral proteins that play important roles in the virus' pathogenesis by enhancing viral replication, survival of the virus within infected cells and/or by facilitating its spread in vivo.

HIV-1 and its Viral Envelope

The outer coat or viral envelope of the HIV-1 virus particle (virion) is composed of several layers of fatty molecules from its host cell and envelope proteins from the virus, which are summatively known as Env. The HIV-1 gp 41 glycoprotein and the HIV-1 gp 120 glycoproteins are the two components of the envelope proteins. HIV-1 gp 120 glycoprotein is required during the initial binding of HIV-1 to its target cell, so a potential approach to disrupt the infectious cycle and to prevent HIV-1 from binding to its host cell would be to block gp 120's binding, for example, with monoclonal antibodies.

Within the viral envelope is the capsid with the viral genetic information. HIV-1 has three structural genes (gag, pol, and env) that encode the enzyme precursors Gag, Pol and Env needed to produce structural proteins for new virus particles.

HIV-1 has, furthermore, several regulatory genes (tat, rev, nef, vif, vpr, and vpu) that contain information needed to produce viral regulatory proteins that control the ability of HIV to infect a cell, produce new copies of the virus or cause disease. The protein encoded by nef, for instance, appears to be necessary for the virus to replicate efficiently, and the vpu-encoded protein influences the release of new virus particles from infected cells.

Two viral regulatory proteins, Tat and Rev, have profound effects on HIV-1's expression, controlling HIV-1 expression at the transcriptional as well as the posttranscriptional level. Rev is an essential protein and promotes the production of structural proteins and infectious virions.

The Tat protein, an early regulatory protein and a nuclear transcriptional activator, is highly important for the virus' replication, since Tat controls proviral DNA transcription to generate the full-length viral mRNA (Kuciak et al., 2008). The Tat protein has a variable length of 86-104 amino acids (aa) and is encoded by two exons; the first exon encodes the first 72 aa (Pugliese et al., 2005; Schwarze et al., 1999). Truncated tat viral protein, for example, Tat 58-72 aa, may also be able to induce the biological affects of the full-length protein. The extracellular form of Tat, which is released from infected cells, is also able to enter target cells and exert its transactivating effects (Ferrari et al., 2003). Mutational analysis of the tat gene of HIV-1 has identified different domains of the protein that are essential or partially essential for Tat-mediated transactivation function (Kuppuswamy et al., 1989).

The HIV-1 proteins Nef, Tat, Vpr and gp120 are also implicated in interfering with macrophage signaling. Nef is a 27-kDa protein that is produced early during infection with HIV-1; it supports viral replication in T cells as well as macrophages and prevents apoptosis of HIV-1-infected T cells (Varin et al., 2003).

HIV-1 replication is tightly regulated at the transcriptional level through specific interactions of the Tat viral regulatory protein, particularly with NF-kappa B, a transcription factor that plays a pivotal role in the activation of genes important for cellular responses to infection and inflammation (Mahlknecht et al., 2008). HIV-1 Tat is a virally encoded transactivating protein, it is essential for virus replication, because it controls efficient transcription of viral genes, and interferes with intracellular signaling. The HIV-1 Tat protein can be detected in the sera of infected patients as well as in the media of infected cells (Ensoli et al., 1993; Westendorp et al., 1995).

Importantly, HIV-1 Tat and HIV-1 gp 120 have also been reported to accelerate activation-induced T cell apoptosis and are so believed to contribute to CD4+ T cell depletion (Westendorp et al., 1995).

### 2G12 Antigens, 2G12-Like Or Non-2G12 Antigens and Antibodies Against Them

HIV-1 gp 120 Glycoprotein

HIV-1 possesses a highly glycosylated surface. The high-mannose clusters of HIV-1's coat protein, the glycoprotein gp120, are specifically recognized by the human monoclonal antibody 2G12. 2G12 is an unusual, domain-exchanged IgG1, which binds to a dense cluster of oligomannose-type glycans on the outer domain of the envelope glycoprotein gp120. Glycosylation with high-mannose type N-glycans is a common feature of the gp120 glycoproteins of different HIV-1 strains.

One of the technical challenges to this investigation is to develop highly sensitive assays to enable detection and characterization of the fine specificity of anti-carbohydrate antibodies in human serum specimens.

2G12 antibody and 2G12-like monoclonal antibodies (mAb)

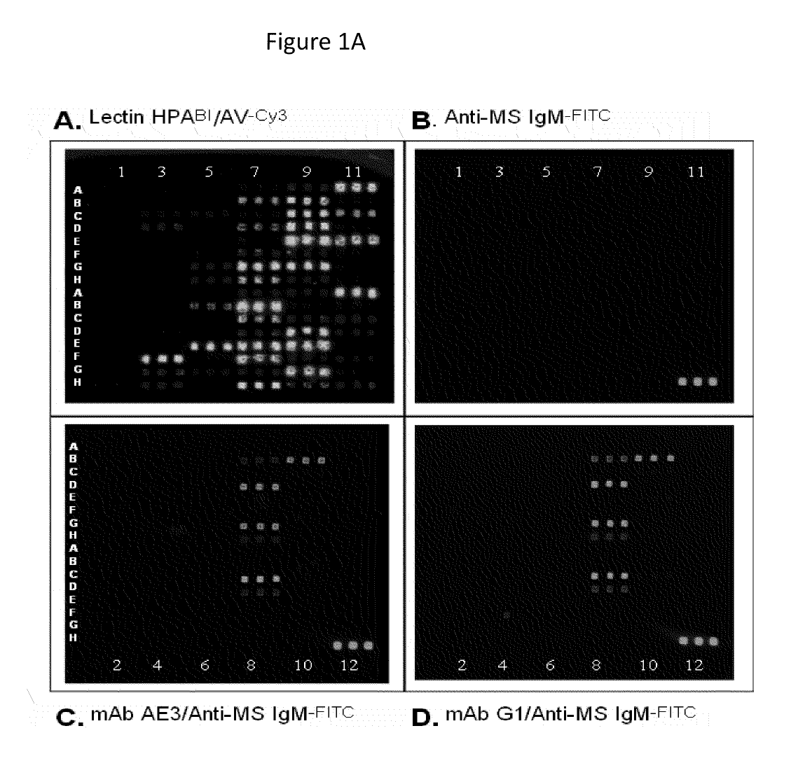

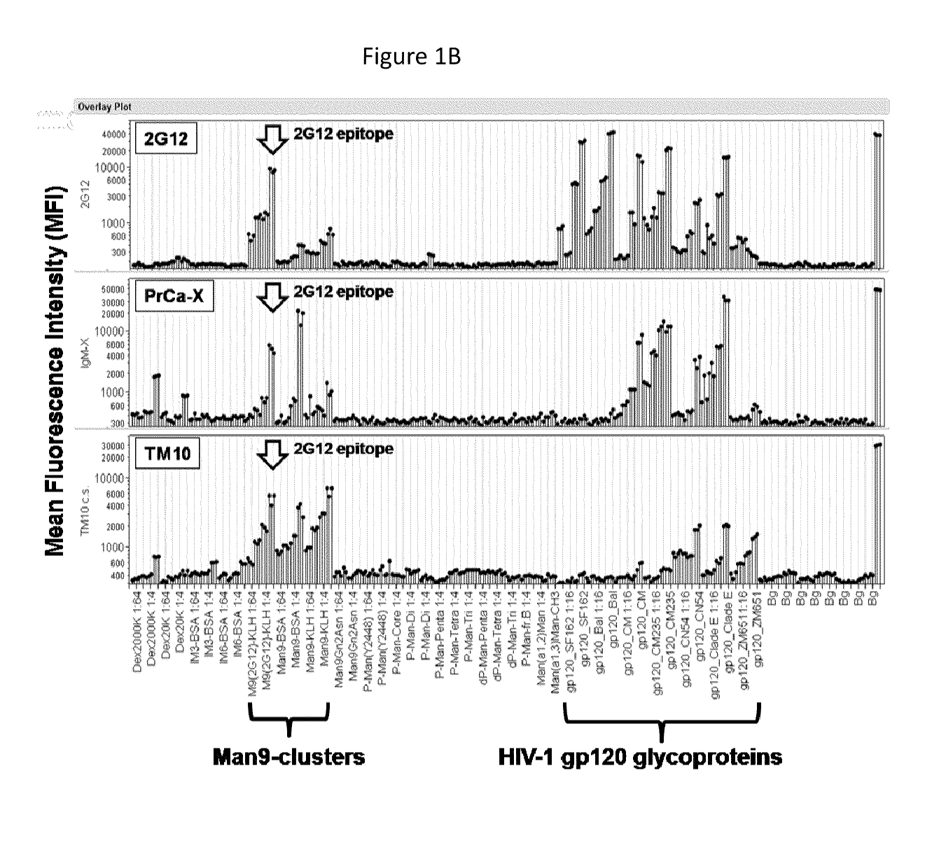

2G12 is an unusual, domain-exchanged IgG1, which binds to a dense cluster of oligomannose-type glycans on the outer domain of the envelope glycoprotein gp120. As defined by carbohydrate microarray analyses (FIG. 1), 2G12 highly and selectively binds to the Man9(2G12)-cluster-KLH conjugate but, to much less extent, to Man9-KLH and Man9-BSA. By contrast, the two 2G12-like mAbs, TM10 and G1(PrCa-X), are not only reactive with Man9(2G12)-cluster-KLH conjugate, but also with the Man9-KLH and Man9-BSA conjugates. Non 2G12-mannose-containing antigens express glyco-epitopes are not recognized by or are poorly reactive with the broadly HIV-1-neutralizing monoclonal antibody 2G12, but are recognized by other antibodies/lectins, such as G1(PrCa-X), TM10, GNA and ConA, which are cross-reactive with HIV-1 glycoproteins. Examples of non 2G12-mannose-containing antigens for which significant antibody activities were detected in HIV-infected human subjects (‘HIV’) and in human control subjects who were not infected with HIV-1 (‘NM’) are found in Table 1: a) Man9-BSA (Row 38); b) P_Man (Row 30); and c) Man2_PAA (Row 34).

Other antigens Examples of other antigens for which differential antibody activities were detected in HIV-infected human subjects (‘HIV’) in comparison to human control subjects who were not infected with HIV-1 (‘NM’) are found in Table 1: a) Carbohydrate antigen Pneumococcus polysaccharide type SIV(PnSIV) (Rows 19, 20); b) Lipid antigen Salmonella typhi lipopolysaccharide (S. typhi_LPS) (Rows 21, 22); c) Protein antigen Tat72R (Tat 1-72 amino acids from first exon) (Row 66); Tat_{—}72R_P181S(Tat72R with a glycine and phenylalanine inserted between amino acid residues 18 and 19) (Rows 69, 70 and 165); and Gag p55 (Rows 85, 86).

Carbohydrate antigen Pneumococcus polysaccharide type SIV(PnSIV) (Rows 19, 20);

b) Lipid antigen Salmonella typhi lipopolysaccharide (S. typhi_LPS) (Rows 21, 22); c) Protein antigen Tat72R (Tat 1-72 amino acids from first exon) (Row 66); Tat_{—}72R_P181S(Tat72R with a glycine and phenylalanine inserted between amino acid residues 18 and 19) (Rows 69, 70 and 165); and Gag p55 (Rows 85, 86).

### HIV-1-Associated Malignancies

HIV-1 infection of CD4⁺ lymphocytes, dendritic cells and macrophages modulates signal transduction pathways and immune response. Although rarely oncogenic itself, HIV-1 exacerbates clinical disease course in individuals who are not only infected with HIV-1, but also with hepatitis C virus, human papilloma virus, Epstein-Ban virus, or human herpesvirus-8.

To date, no effective prophylactic or therapeutic vaccines to prevent or cure HIV-1 infection are available. Combinations of antiretroviral drugs reduce perinatal infection and HIV-1 associated morbidity and mortality. Increased survival among HIV-1 infected adults and children could lead to an increase in malignancies due to the state of chronic immune suppression, coinfection by oncogenic viruses, and/or persistent HIV-1 infection.

Non-Hodgkin's lymphomas (NHLs) are a diverse group of hematologic cancers which encompass any lymphoma other than Hodgkin's lymphoma. An lymphoma is a cancer that originates from lymphocytes, which are a subpopulation of white blood cells. The occurrence of non-Hodgkin's lymphomas can be increased in a state of chronic immune suppression, as it is the case with HIV-1-infection, and therefore non-Hodgkin lymphomas can be considered as HIV-1-associated malignancies.

### Antibody Types, Fragments, Production and Detection

Antibodies which are also known as immunoglobulines (Ig) are, in their most abundant form of immunoglobulin G (IgG), usually heterotetrameric glycoproteins that are composed of two identical heavy and two identical light chains, whereby each light chain is linked to a heavy chain by one covalent disulfide bond. Each heavy chain has at one end a variable domain followed by a number of constant domains. Each light chain has a variable domain at one end and a constant domain at its other end. The constant domain of the light chain is aligned with the first constant domain of the heavy chain and the variable domain of the light chain is aligned with the variable domain of the heavy chain.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. Rather, it is concentrated in three segments called complementarity-determining regions (CDRs) or hypervariable regions. The more highly conserved portions of variable domains are called the framework. The CDRs in each chain are held together in close proximity by the framework regions and, with the CDRs from the other chain, contribute to the formation of the antigen-binding site of antibodies. Collectively, the six CDRs confer antigen-binding specificity to the antibody. However, even a single variable domain has the ability to recognize and bind antigen, although at a lower affinity than the entire binding site. The constant domains are not directly involved in binding an antibody to an antigen, but exhibit various effector functions.

Antibodies can be digested with the enzyme papain into two identical antigen-binding fragments called “Fab” fragments, each with a single antigen-binding site and a residual, readily crystallizable “Fc” fragment. Pepsin treatment yields an F(ab)2 fragment that has two antigen-combining sites and is still capable of cross-linking antigen.

“Fv” is the minimum antibody fragment which contains a complete antigen-recognition and antigen-binding site. This region consists of a dimer of one heavy-chain and one light-chain variable domain in tight association.

Polyclonal antibodies are immunoglobulins of different specificities and originate from different B cells. They are a mixture of immunoglobulin molecules secreted against a specific antigen, each recognizing a different epitope. While polyclonal antibodies are easier to obtain, they are usually raised in animals such as rabbits and horses, they are likely specific for more than one epitope of an antigen.

Monoclonal antibodies, in contrast, are produced by the same B cell clone and are, therefore, identical copies of the same immunoglobulin; they are highly specific against a particular epitope. Monoclonal antibodies are typically made by fusing myeloma cells with the spleen cells from a mouse that has been immunized with the desired antigen.

Humanized antibodies or chimeric antibodies are types of monoclonal antibodies that have been synthesized using recombinant DNA technology to circumvent the clinical problem of immune response to foreign antigens. The standard procedure of producing monoclonal antibodies yields mouse antibodies. Although murine antibodies are very similar to human ones in their immunoglobulin structures, there are xenogenic to human hosts, and the human immune system recognizes mouse antibodies as foreign, rapidly removing them from circulation and causing systemic inflammatory effects.

Humanized antibodies are produced by merging the DNA that encodes the binding portion of a monoclonal mouse antibody with human antibody-producing DNA. One then uses mammalian cell cultures to express this DNA and produce these half-mouse and half-human antibodies that are not as immunogenic as the murine variety.

In certain embodiments, the antibody is immobilized on a solid phase, e.g. for diagnostic assays. For diagnostic uses, a labeled antibody (e.g. antibody bound to a detectable label) might be used; the labeling can be direct (i.e., physically linked) or indirect. Detectable labels can be fluorescers, radioisotopes, enzymes, chemiluminescers or other labels for direct detection. Examples of indirect labeling include detection of a primary antibody using a fluorescently labeled secondary antibody and end-labeling a DNA probe with biotin such that it can be detected with fluorescently labeled streptavidin.

Antibody detection can be achieved using various methods, including flow cytometry, microscopy, radiography, scintillation counting, immunoassays, which are all well established and known in the art.

Immunoglobulin M or IgM, is a primary antibody isotype that is present on surfaces of B cells and produced by B cells. IgM antibodies are involved in the primary response upon the exposure to an antigen and appear early in the course of an infection and usually reappear, to a less extent, after further exposure. IgM also plays an important role in antibody-dependent cell-mediated cytotoxicity (ADCC).

Immunoglobulin G (IgG) is the most abundant immunoglobulin and synthesized and secreted by B cells. IgG antibodies are predominately involved in the secondary immune response. Only IgG can pass through the human placenta, thereby providing protection to the fetus in utero. IgG can bind to many different pathogens and protects the body against them by agglutination and immobilization, complement activation, phagocytosis and neutralization of their toxins. IgG also plays an important role in antibody-dependent cell-mediated cytotoxicity (ADCC).

### Therapeutic Use of Antibodies

Monoclonal antibodies as well as their fragments or derivates that bind only to cell-specific or microbe-specific carbohydrate antigens or glyco-epitopes and, as a consequence, induce an immunological response against those targeted cells or microbes have become a viable option in therapeutic approaches such as cancer therapy or passive vaccination for prophylactic or therapeutic purposes.

Therapeutic monoclonal antibodies can exert their anti-tumor effects through antibody-dependent cellular cytotoxicity and complement-dependent cytotoxicity with IgM antibodies being the most efficient isotype for complement activation.

Direct therapeutic applications of monoclonal antibodies against a carbohydrate antigen or glyco-epitope are often based on the systemic administration of such antibodies, their fragments or their synthetic derivatives to patients, once the presence of the particular antigen has been confirmed. The time course of the therapeutic effect typically correlates directly with the residence time and/or remaining concentration of such antibodies in the body; therefore, repeated or chronic administration is often necessary.

Another immunotherapy approach is based on the selective activation of the immune system of patients to combat and eliminate abnormal cells and intruded pathogens before they can spread and cause a disease.

### Pharmaceutical Composition for Vaccination and Utility

The identification of antigens from HIV-1 virions that elicit immune responses and that can potentially be utilized to formulate a pharmaceutical vaccination product to prophylatically immunize human subjects who are not yet infected with HIV-1 is a key aspect of the present invention. A further important aspect of the present invention is the use of such identified antigens from HIV-1 virions in a therapeutic vaccination product to provide and to enhance immune response in subjects who are already infected with HIV-1. Table 1 shows several antigens that elicited a clearly distinguishable immune response in human subjects who were already infected with HIV-1 (‘HIV’) compared to human control subjects who were not infected with HIV-1 (‘NM’). Antigens that elicited a significantly different immune response between HIV and NM are a) carbohydrate antigens PnSIV (rows 19, 20); Man9-BSA (row 38); b) lipid antigen S. typhi LPS (rows 21, 22); c) protein antigens from HIV-1 virions with point mutations: Tat72R (row 66); Tat_{—}72R_P181S (rows 69, 70 and 165); and Gag p55 (rows 85, 86).

In one embodiment, the present invention relates to a pharmaceutical composition for vaccinating a mammalian subject against HIV-1 and/or HIV-1-associated malignancies comprising either high-mannose clusters (active immunization) or at least one monoclonal antibody such as 2G12, TM10 or G1(PrCa-X) that recognizes high-mannose clusters and high-mannose-carrier protein conjugates that are characteristic of HIV-1 and/or HIV-1-associated malignancies (passive immunization). The pharmaceutical composition comprising either high-mannose clusters (active immunization) or at least one antibody in accordance to the use of the present invention may be administered as a vaccine with various pharmaceutically acceptable carriers that are commonly used in the formulation of vaccines. Pharmaceutically acceptable carriers include those approved for use in animals and humans and include but are not limited to diluents as well as adjuvants such as water, oils, saline, dextrose solutions, glycerol solutions and excipients such as starch, glucose, lactose, sucrose, gelatin, malt, rice, flour, chalk, silica gel, sodium stearate, glycerol monostearate, talc, sodium chloride, powdered non-fat milk, propylene glycol and ethanol. Pharmaceutical compositions may also include emulsifying agents or pH buffering compounds.

In one embodiment, the present invention relates to a pharmaceutical composition for vaccinating a human subject against HIV-1 and/or HIV-1-associated malignancies comprising at least one monoclonal anti-Tat antibody directed against an antigenic determinant on the HIV-1 Tat protein (passive immunization). The pharmaceutical composition comprising the at least one monoclonal anti-Tat antibody in accordance to the use of the present invention may be administered as a vaccine with various pharmaceutically acceptable carriers that are commonly used in the formulation of vaccines. Pharmaceutically acceptable carriers include those approved for use in animals and humans and include but are not limited to diluents as well as adjuvants such as water, oils, saline, dextrose solutions, glycerol solutions and excipients such as starch, glucose, lactose, sucrose, gelatin, malt, rice, flour, chalk, silica gel, sodium stearate, glycerol monostearate, talc, sodium chloride, powdered non-fat milk, propylene glycol and ethanol. Pharmaceutical compositions may also include emulsifying agents or pH buffering compounds.

In another embodiment, the present invention relates to a pharmaceutical composition for vaccinating a human subject against HIV-1 and/or HIV-1-associated malignancies comprising either high-mannose clusters (active immunization) or at least one monoclonal antibody such as TM10 or G1(PrCa-X) that recognizes high-mannose clusters and high-mannose-carrier protein conjugates that are characteristic of HIV-1 and/or HIV-1-associated malignancies (passive immunization). The pharmaceutical composition comprising either high-mannose clusters (active immunization) or at least one antibody in accordance to the use of the present invention may be administered as a vaccine with various pharmaceutically acceptable carriers that are commonly used in the formulation of vaccines. Pharmaceutically acceptable carriers include those approved for use in animals and humans and include but are not limited to diluents as well as adjuvants such as water, oils, saline, dextrose solutions, glycerol solutions and excipients such as starch, glucose, lactose, sucrose, gelatin, malt, rice, flour, chalk, silica gel, sodium stearate, glycerol monostearate, talc, sodium chloride, powdered non-fat milk, propylene glycol and ethanol. Pharmaceutical compositions may also include emulsifying agents or pH buffering compounds

A composition of the present invention is typically administered parenterally in dosage unit formulations containing standard, well-known non-toxic physiologically acceptable carriers, adjuvants, and vehicles as desired. The term ‘parenteral’, as used herein, includes intravenous, intramuscular, intraarterial injection, or infusion techniques. Among the acceptable vehicles and solvents that may be employed are water, Ringer's solution, and isotonic sodium chloride (saline) solution. In addition, sterile oils are conventionally used as a solvent or suspending medium.

The compositions of the invention are administered in substantially non-toxic dosage concentrations sufficient to ensure the release of a sufficient dosage unit into a human subject to provide the desired therapeutic immunity. The actual dosage administered and the frequency of dosage administration (vaccinating) will be determined by physical and physiological factors such as age, body weight, severity of condition and/or clinical history of the human subject.

The therapeutic efficacy of the vaccination can be determined by a comparison of biological samples such as serum taken some time before and after the vaccinating step, using at least one (monoclonal) antibody that recognizes high-mannose clusters and high-mannose-carrier protein conjugates that are characteristic of HIV-1 and/or HIV-1-associated malignancies. A decrease in detected HIV-1 or HIV-1-associated malignancy or a portion thereof in biological samples such as serum taken after the vaccinating step, in comparison to biological samples taken before the vaccinating step, indicates therapeutic efficacy of the vaccination. Further vaccinating steps might be undertaken, as determined by the degree and sustainability of the efficacy of the vaccination.

### Carbohydrate Microarrays

In the field of glycomics research, carbohydrate microarrays are high-throughput discovery tools on a biochip platform which are useful for identifying immunologic sugar moieties, including complex carbohydrates of cancer cells and sugar signatures of microbial pathogens. Within the field of immunology, carbohydrate microarrays are important tools to investigate the antigenic diversity of carbohydrate antigens. Carbohydrate microarrays can be designed as natural and/or synthetic mono-, di-, oligo- or polysaccharide chips as well as glycoconjugate chips. Carbohydrate cluster arrays are able to identify and target immunogenic sugar moieties such as polysaccharides, glycoproteins, glycolipids, glycoconjugates and glyconeoconjugates. Details of an exemplary protocol are published by one of the inventors (Wang et al., 2002; 2004; 2007).

High-Mannose (Man9) Clusters

N-glycan cores and internal chains are usually cryptic, i.e, they are masked by other sugar moieties such as a) high mannose chains (Man clusters), b) triantennary type II (Galβ1→4G1cNAc chains (Tri-II), c) multivalent type II chains (m-II), or d) agalactosyl-Tri-/m-II glycol-epitopes (Tri/m-Gn).

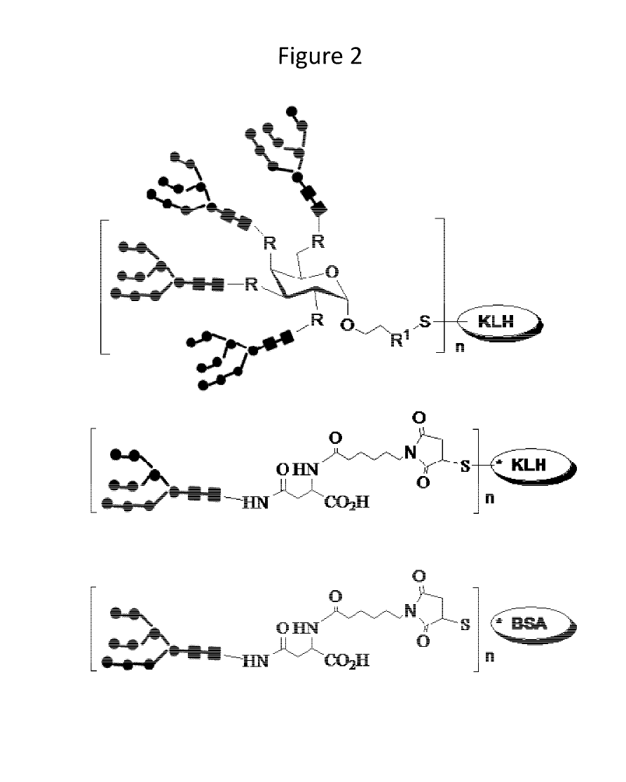

High-mannose (Man9)-clusters occur in different structural configurations. FIG. 2 shows a) [(Man9)4]n-KLH, which a 2G12-specific glyco-epitope; b) (Man9)n-KLH, which is poorly reactive with 2G12 but highly reactive with TM10 and G1(PrCa-X) and thus display the Tm10 and G1(PrCa-X) epitopes on a KLH carrier; and c) (Man9)n-BSA, which display TM10 and G1(PrCa-X)—epitopes on a BSA carrier molecule.

### Disease Detection and Disease Monitoring/Prognostics

The presence of HIV-1 or HIV-1-associated malignancies can be detected using anti-high mannose (Man9) antibodies against the corresponding glyco-epitopes; this approach allows differential diagnosis and also allows to prognostize disease outcome. This can be achieved with carbohydrate cluster arrays, as described above, immunoassays against carbohydrates as well as flow cytometry-based multiplex bead assays, particularly FACS-based multiplex bead assays.

Flow Cytometry

Flow cytometry is a technique for counting and examining small particles such as cells by suspending them in a stream of fluid and passing them by an electronic detection apparatus. It allows simultaneous multiparametric analysis of the physical and/or chemical characteristics of each individual particle or cell. Briefly, a beam of light (usually laser light) of a single wavelength is directed onto a hydrodynamically-focused stream of fluid. A number of detectors are aimed at the point where the stream passes through the light beam: one in line with the light beam (forward scatter), several in perpendicular position (side scatter) and at least one fluorescence detector. Each suspended cell (from 0.15 μm-150 μm) passing through the light beam scatters the light in some way, and fluorescent molecules (naturally occurring or as part of an attached label or dye) may be excited into emitting light at a longer wavelength than the light source. This combination of scattered and fluorescent light is recorded by detectors. The forward scatter correlates with the cell volume, while the side scatter depends upon the inner complexity of the cell (such as shape of the nucleus). The data generated by flow cytometers can be plotted in a single dimension to produce a histogram or in two-dimensional or three dimensions plots. The regions on these plots can be sequentially separated, based on fluorescence intensity, by creating a series of subset extractions, termed “gates.” Specific gating protocols exist for diagnostic and clinical purposes, especially for hematology. There are also flow cytometers who only use light scatter, without fluorescence, for the analysis.

Fluorescence activated cell sorting (FACS) is a specialized type of flow cytometry and provides a method of sorting a heterogeneous mixture of cells into two or more containers, a single cell at a time, based upon the specific light scattering and fluorescent characteristics of each cell. The use of multicolor, multiparameter FACS requires primary conjugated antibodies at defined fluorophore-to-protein (FTP) ratios.

### Prophylactic and Therapeutic Intervention Strategies

It is one of the objectives of the present invention to describe prophylactic intervention strategies in the case of HIV-1 infectious disease and HIV-1-associated malignancies using anti-high mannose (Mang)-based HIV-1 vaccines and/or vaccines against HIV-1-associated malignancies.

An antigen/antibody-based intervention can be an active immunization or vaccination or a passive immunization or vaccination. In case of an active vaccination strategy, a human subject's immune response is induced by the administration of a pharmaceutical composition that contains a defined amount of high mannose cluster carbohydrate antigens or glyco-epitopes that are part of a glycan that is characteristic of HIV-1 or an HIV-1-associated malignancy and against which the human subject's organism is induced to produce a humoral immune response, i.e. based on production of antigen-specific antibodies by the activated B cells, to with or without T cell activation. The resulting immunity is typically long-lasting and might require repeated (booster) vaccinations at defined timepoints (after several weeks or months or years) to maintain the desired level of immunity.

In case of a passive vaccination strategy, a human subject's immune response is induced by the administration of a pharmaceutical composition that contains a defined amount of monoclonal antibodies that have been raised against particular high mannose cluster carbohydrate antigens or glyco-epitopes that are part of a glycan that is characteristic of HIV-1 or an HIV-1-associated malignancy.

Therapeutic intervention in HIV-1 and HIV-1-associated malignancies with anti high-mannose (Man9)-based HIV-1 vaccines and anti high-mannose (Man9)-based vaccines targeting HIV-1-associated malignancies.

Humanized and fully human monoclonal antibodies are currently used to pharmacologically intervene and combat HIV-1 viruses. Furthermore, broadly neutralizing monoclonal antibodies such as 2G12 have been tested against HIV-1 infection. Therapeutic intervention with anti high-mannose (Man9)-based vaccines is a relatively new, but very promising approach.

### UTILITY

The present invention will be useful for the diagnosis, monitoring and prognostics of HIV-1 and HIV-1-associated malignancies by detecting and identifying antibodies against glyco-epitopes characteristic for HIV-1 and/or HIV-1-associated malignancies.

The present invention will, furthermore, be useful for the therapeutic intervention in HIV-1 and HIV-1-associated malignancies via active or passive immunization/vaccination using Man9-conjugate based HIV-1 vaccines, alone or in combination with a FDA approved BCG (TB) vaccine. As of 2005, the Center of Disease Control (CDC) estimated that 9% of all tuberculosis cases and nearly 16% of tuberculosis cases among persons aged 25 to 44 were occurring in HIV-infected persons, particularly in HIV-1-infected individuals. HIV-1 infected individuals who are also infected with Tubercle Bacillus (TB) are a very high risk group for developing active, potentially contagious TB disease due to their seriously weakened immune system. Therefore, it would be highly beneficial to develop HIV-1 vaccines in combination with a vaccine against TB. Furthermore, the inventors of the pre sent invention have observed that the administration of vaccines that contain inactivated TB can contribute to the induction of anti-Man9 antibodies in human subjects.

The present invention may also be useful for preventing HIV-1 infection and/or the development of HIV-1-associated malignancies. To date, no vaccine to prevent HIV-1 or HIV-1-associated malignancies exists. Antiretroviral therapy is currently the treatment of choice for individuals who have been diagnosed with HIV-1. However, antiretroviral therapy is not effective in preventing the spread of HIV-1 among still healthy individuals or individuals with undiagnosed HIV-1 infections. Therefore, a vaccine against HIV-1 infection and/or HIV-1-associated malignancies would be most likely, and perhaps the only way by which a further spread of HIV-1 infections and development of HIV-1-associated malignacies can be prevented.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### EXPERIMENTAL PROCEDURES

The following methods and materials were used in the examples that are described further below.

Printing Protein and Carbohydrate Microarrays

A high-precision robot designed to produce cDNA microarrays (PIXSYS 5500C, Cartesian Technologies, Irvine, Calif.) was utilized to spot antigen preparations, including proteins/peptides and carbohydrates of various composition onto SuperEpoxy 2 Protein slides (Arrayit Corporation, Sunnyvale, Calif., USA). Proteins and carbohydrates were dissolved in PBS (pH 7.4) and saline (0.9% NaCl), respectively, and printed with spot sizes of ˜150 μm and at 375 μm intervals, center to center. The printed microarrays were air-dried and stored at room temperature without desiccant before application.

Although the SuperEpoxy 2 substrate requires amino-containing protein or amino-modified DNA, carbohydrate and lipid antigens without amino-substituents were stably immobilized, as detailed herein.

Microarray Data-Processing, Standardization and Statistic Analysis.

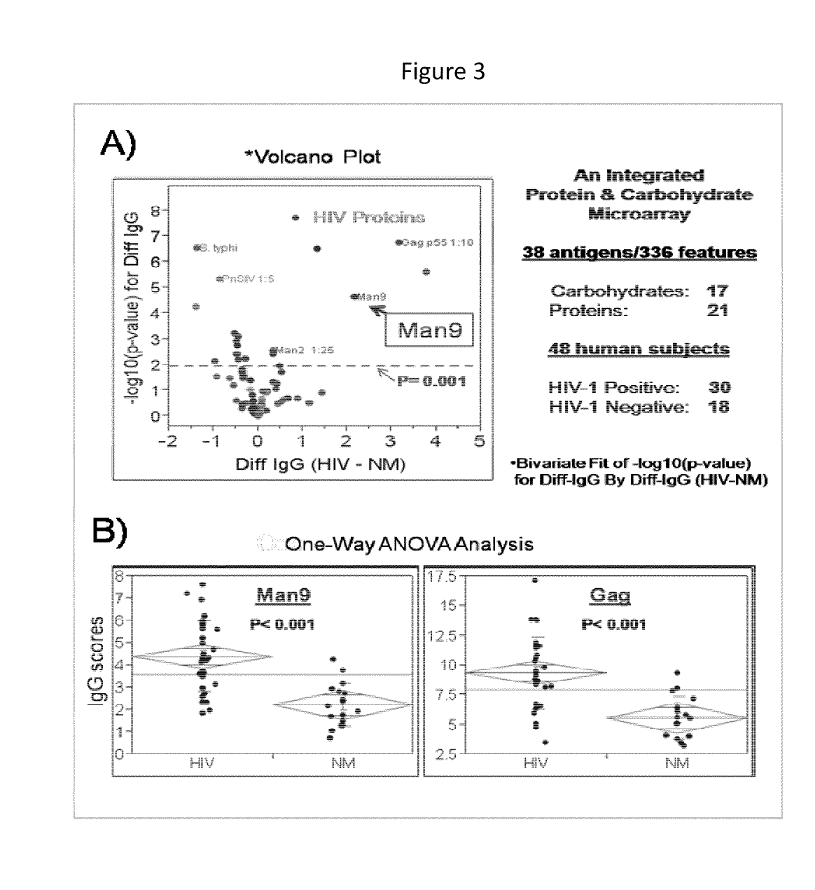

Fluorescence intensity values for each array spot and its background were calculated using ScanArray Express software. SAS Institute's JMP-Genomics software package (http://www.jmp.com/) was applied for microarray data standardization and statistic analysis. The Relative Antibody Reactivity (RAR) scores specified in FIG. 3 were defined as the log 2 transformed and IQR standardized microarray values. The IQR function in JMP-Genomics normalizes array datasets by setting their interquartile ranges (IQR) to be identical, which is essential for comparison of the internally standardized RAR scores among experimental groups. An antigen-by-antigen ANOVA model was applied to obtain statistically significant differences between groups in comparison. Data from triplicate spots for each antigen were included in the ANOVA model for that antigen. A cut-off to detect significant differences is determined by applying a multiple testing correction to statistical results from the ANOVA model.

The preparation of [(Man9)4]n-KLH and (Man9)n-KLH was previously described (Wang et al., 2004; Ni et al., 2006). The (Man9)n-KLH and (Man9)n-BSA were synthesized by conjugating KLH-SH or BSA-SH with maleimide-functionalized Man9. The carbohydrate contents in the glycoconjuates (Man9)n-KLH and (Man9)n-BSA were about 15%.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, parts are parts by weight, molecular weight is average molecular weight, temperature is in degrees Centigrade, and pressure is at or near atmospheric.

### Example 1

### Detection of Significant Levels of Anti-High-Mannose-Cluster Antibodies in HIV-1 Infected Human Subjects Using Carbohydrate Cluster Arrays

In this experiment, a panel of thirty-eight antigens, including proteins (n=21) and carbohydrate antigens (n=17), was spotted in the superepoxy2 bioarray substrate. This technology supports simultaneous measurement of the relative antibody reactivities with distinct antigenic structures of an infectious agent, such as the Gag p55 protein and high-mannose-clusters of HIV-1. Using these arrays, 30 HIV-1 infected human subjects and 18 HIV-1 negative control subjects were analyzed. The results are summarized in FIGS. 3and 4 as well as in Table 1 (see Appendix A), which shows the antigens that detected significantly different antibody activities in HIV-1-infected human subjects (HIV) in comparison to non-infected human control subjects (NM), highlighted in bold. These include a) carbohydrate antigen PnSIV (Rows 19, 20); Man9-BSA (Row 38); b) lipid antigen S. typhi_LPS(Raw 21, 22); c) Protein antigen Tat72R(Raw 66); Tat_{—}72R_P181S (Rows 69, 70 and 165); and Gag p55 (Rows 85, 86). A panel of thirty-eight antigenic structures was spotted in a versatile bioarray substrate. FIG. 3A is a “Volcano Plot” produced by JMP software package for a comparative analysis of microarray datasets. In this case, it compares the profiles of antigen-specific IgG antibodies in circulations between the HIV-1-infected subjects (HIV) and non-infected controls (NM). The X-axis displays differences (log 2 ratios) between the two groups, where a difference of 1 is approximately a twofold change in IgG signal detected by microarrays. The Y-axis shows the levels of statistic significance as −log 10 (p-value) for the comparison between the two groups. The dashed red line is the value for significance with the correction for false discovery rate. FIG. 3B shows One-Way ANOVA analysis of two probes, Man9 and Gag. Both detected significantly elevated IgG antibodies in the HIV-1 positive group (p<0.001). Array datasets were processed and statistically analyzed using SAS Institute's JMP-Genomics 3.2 software package. Each dot in FIG. 3b is the mean RAR Score* of triplicate array detection of a subject. The p-values in each panel indicate the statistical reliability of the difference in means between groups (green bar) while the standard deviations (green diamond around the mean value) indicate the size of the difference, in relation to the distribution of values in each group. FIG. 3b was generated by plotting the mean RAR scores of each group.

This microarray analysis revealed a number of positive probes that detected significant amounts of antigen-specific IgG antibodies in the HIV-1-infected subjects. There are expected protein probes, such as the Gag p55 of HIV-1. Importantly, this assay also detected significant amounts of anti-Man9 antibodies in the group of HIV-1 infected subjects. In this microarray design, we considered the need of specificity assignment by spotting a panel of probes with certain structural similarity, such as the mannose-containing carbohydrate antigens Man9-BSA, Man2-polyacrylamide (PAA), Yeast phosphomannan Y2448 (P-Man) and Lipoarabinomannan (LAM).

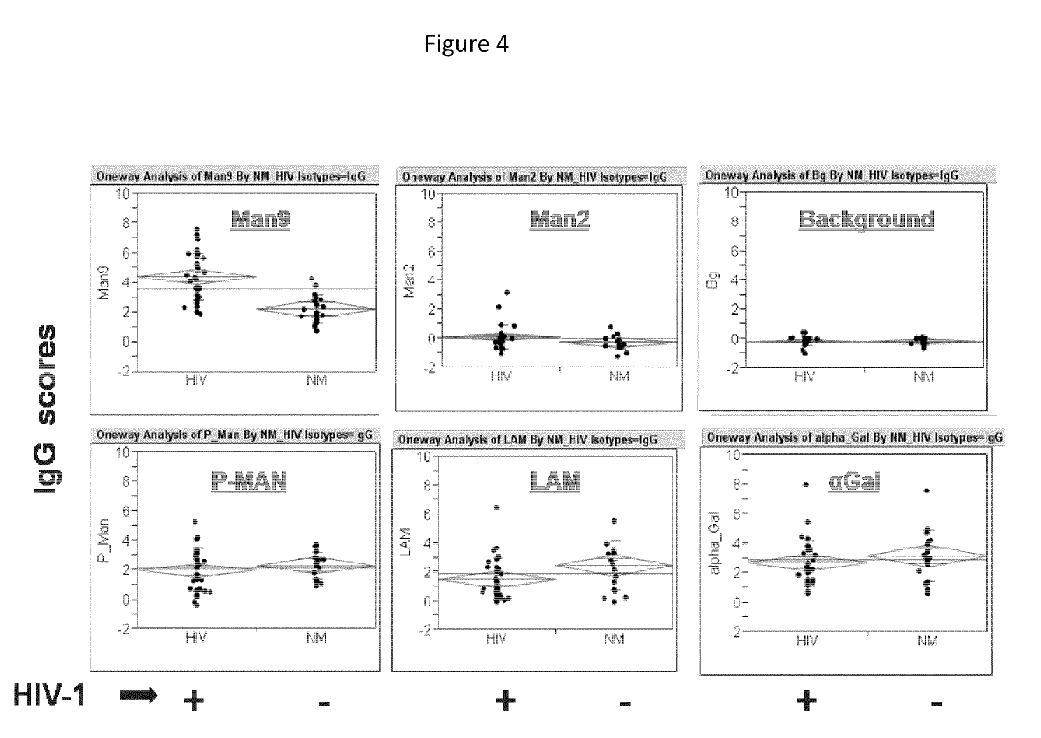

FIG. 4 is a comparison of the relative antibody reactivities of the two groups of human subjects (HIV-1-infected subjects (HIV) and non-infected controls (NM)) with five probes, including the four mannose-containing antigens Man9-BSA, Man2-PAA, P-Man and LAM and an alpha-Gal antigen. The latter detects abundant amounts of natural antibodies in human circulation and, thus, serves as a positive control for comparing the levels of antibody detection by this microarray assay. It is clearly shown that the Man9-BSA probe detected similar levels of serum IgG as those detected by P-Man, a yeast phosphomanan (Y2448), and those captured by LAM in the control group. However, only the levels of the anti-Man9 IgG antibodies are significantly higher than those detected in the control group. In addition, the amount of anti-Man9 IgG in the HIV-1 positive group is markedly higher than the amounts of anti-alpha Gal antibodies in both the HIV-1-infected (HIV) and non-infected groups (NM). Furthermore, in the same assay significantly increased anti-Man9 IgM antibodies were detected in the HIV-1 infected subjects. This demonstrated that detection of Man9-specific serum antibodies is highly correlated to the status of HIV-1 infection in the investigated human subjects.

### Example 2

### Immunization of Mice with a Glycoconjugate Bearing Man9-Clusters Elicited Antibodies that are Highly Cross-Reactive with HIV-1 gp 120 Glycoproteins

It was further examined whether a glyco-conjugate that displays the high-mannose-clusters is able to elicit anti-HIV-1 gp120 antibodies in vivo. We coupled Man9 units (Man9GlcNAc2) with a protein carrier keyhole limpet hemocyanin (KLH) and immunized SJL/J mice in the presence and absence of myelin peptide PLP_{139-151.} The latter is a potent autoimmune T-cell activator in the SJL/J background and was used to enhance anti-glycan immune response by breaking the immune tolerance with autoantigens. Serum antibodies pre- and post-immunization were then characterized by microarrays spotted with a panel of HIV-1 gp120 proteins derived from multiple strains/clades of HIV-1.

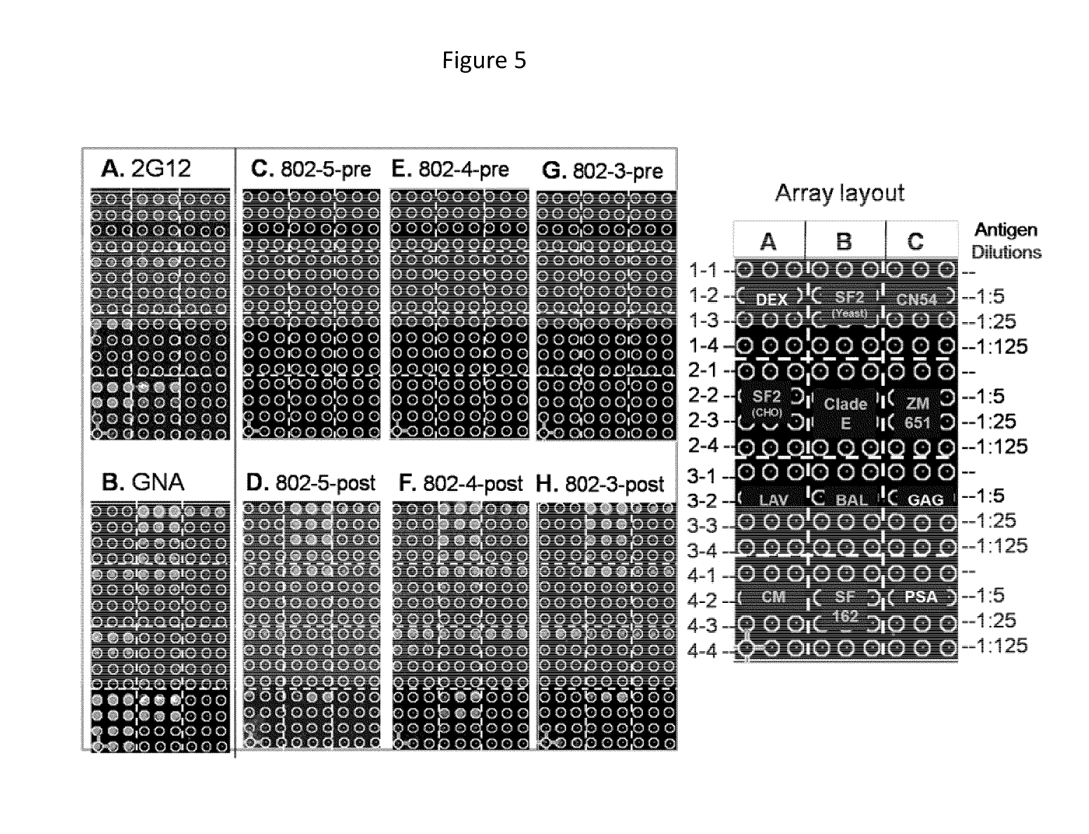

Preservation of carbohydrate-based HIV-1 neutralization epitopes in this bioarray was verified by staining the arrays with human mAb 2G12 (FIG. 5A) and lectin GNA (FIG. 5B). Sera were scanned by this array for antibody reactivities with a panel of twelve antigens. Array location and series dilutions of corresponding antigens are shown in the right column of FIG. 5. Visual inspection of microarray images readily revealed that a single injection of the Man9-KLH-containing emulsion led to induction of IgG antibodies that are highly reactive with HIV-1 gp120 glycoproteins.

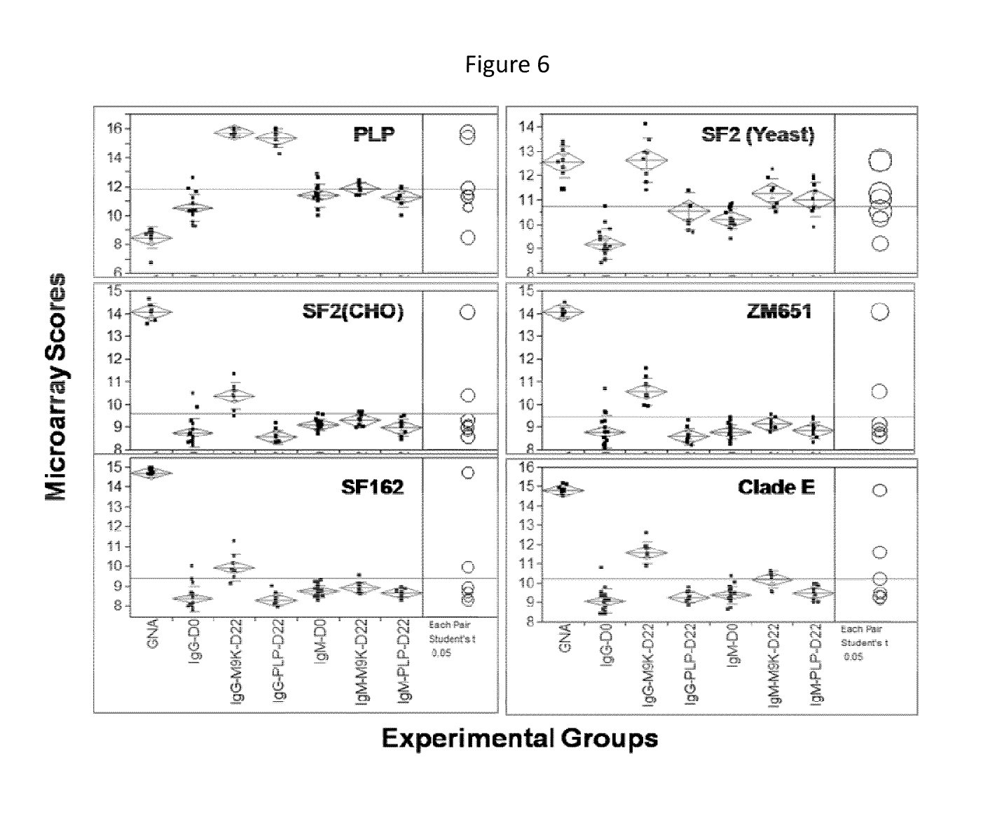

FIG. 6 illustrates quantitatively both IgG and IgM antibody responses to different antigens under each immunization condition. In the upper left panel, the PLP-specific antibodies were determined, which shows induction of anti-PLP IgG but not IgM responses in the two immunization group, i.e., Man9-KLH plus PLP and PLP alone. The rest of panels in the Figure illustrate antibody responses to corresponding gp120 glycoproteins of HIV-1. The Man9-KLH plus PLP immunization but not PLP alone group induced significant amounts of anti-gp120 IgG antibodies and, to a less extent, anti-gp120 IgM antibodies.

### Example 3

### Two Prostate-Cancer-Targeting Mabs (TM10 and G1(PrCa-X)) Illustrate Overlapping Glycan-Binding Profiles of 2G12 and are Significantly Cross-Reactive with the HIV-1 gp120 Glycoproteins”

Glycan binding profiles of three monoclonal antibodies (mAbs 2G12, G1(PrCa-X) and TM10) and their cross-reactivities with HIV-1 gp120 glycoproteins were characterized using the described carbohydrate microarrays. G1(PrCa-X) and TM10 were obtained by cancer immunizations. 2G12 is one of the best characterized anti-Man9 mAb. Results shown in FIG. 1 revealed: a) All three mAbs are highly reactive with a well-defined 2G12-glyco-epitope (marked in the figure); b) All three mAbs are highly cross-reactive with HIV-1 gp120 glycoproteins spotted on the arrays; c) 2G12 differs from G1(PrCa-X) and TM10 in having the highly selective binding to the 2G12-glyco-epitope and lower reactivities with Man9-KLH and Man9-BSA; d) the three mAbs show different binding patterns in reacting with the seven preparations of HIV-1 gp120 glycoproteins.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Ensoli B et al. (1993). Release, uptake and effects of extracellular human Immunodeficiency virus type 1 Tat protein on cell growth and viral transactivation. J Virol 67:277-287.
Ferrari A et al. (2003). “Caveolae-mediated internalization of extracellular HIV-1 Tat fusion proteins visualized in real time.” Mol Ther 8: 284-294.
Kuciak M et al. (2008). The HIV-1 transcriptional activator Tat has potent nucleic acid chaperoning activities in vitro. Nucl. Acids Res. 36: 3389-3400.
Kuppuswamy M et al. (1989). Multiple functional domains of Tat, the trans-activator of HIV-1, defined by mutational analysis. Nucleic Acids Res 17:3551-3561.
Mahlknecht U et al. (2008). NF-κB-dependent control of HIV-1 transcription by the second coding exon of Tat in T cells. J Leukoc Biol 83:718-727.
Ni J et al. (2006). Toward a carbohydrate-based HIV-1 vaccine: synthesis and immunological studies of oligomannose-containing glycoconjugates. Bioconjug Chem 17:493-500.
Pugliese A et al. (2005). “A review of HIV-1 Tat protein biological effects.” Cell Biochem Funct 23:223-227.
Schwarze S R et al. (1999). “In vivo protein transduction: delivery of a biologically active protein into the mouse.” Science 285:1569-1572.
Varin A et al. (2003). Exogenous Nef protein activates NF-kappa B, AP-1, and c-Jun Nterminal kinase and stimulates HIV transcription in promonocytic cells. Role in AIDS pathogenesis. J Biol Chem 278:2219-2227.
Wang D et al. (2002). Carbohydrate microarrays for the recognition of cross-reactive molecular markers of microbes and host cells. Nat Biotechnol 21:275-281.
Wang D et al. (2004). Glycan arrays lead to the discovery of autoimmunogenic activity of SARS-CoV. Physiol. Genomics 18:245-249

Wang D et al. (2007). Photo-generated glycan arrays identify immunogenic sugar moieties of Bacillus anthracis exosporium. Proteomics 7:180-184.
Wang L X et al. (2004). Binding of high-mannose-type oligosaccharides and synthetic oligomannose clusters to human antibody 2G12: implications for HIV-1 vaccine design. Chem Biol 11:127-134.
Westendorp M O et al. (1995). Sensitization of T cells to CD95-mediated apoptosis by HIV-1 Tat and gp 120. Nature 375:497-500.

What is claimed is:

1. A carbohydrate microarray comprising & displaying a multitude of structural and configural variants of oligomannosyl (Man5-9G1cNAc2)-clusters and detecting oligomannosyl-cluster-specific antibodies that are characteristic for HIV-1 infection or a portion thereof in human subjects or in an animal model of AIDS.

2. A method of detecting a carbohydrate antigen or glyco-epitope characteristic of HIV-1 or a portion thereof in a biological sample according to claim 1, the method comprising detecting at least one anti-oligomannosyl cluster antibody.

3. A method of detecting a carbohydrate antigen or glyco-epitope characteristic of an AIDS-associated malignancy in a biological sample, the method comprising detecting at least one anti-oligomannosyl cluster antibody.

4. A pharmaceutical composition for passive immunization in a human subject against HIV-1 infection, the composition comprising at least one monoclonal anti-high mannose (Man9) antibody directed against a high mannose cluster on HIV-1 envelope glycoprotein, gp120, and/or on HIV-1 infected cells.

5. The pharmaceutical composition according to claim 4, wherein the monoclonal antibody is a humanized antibody.

6. The pharmaceutical composition according to claim 4, wherein the monoclonal antibody is a fully human antibody.

7. A method for inducing a therapeutic immunity against HIV-1 infection, the method comprising administering the pharmaceutical composition of claim 4 to a human subject.

8. A microarray comprising displaying a multitude of structural and configural variants of HIV-1 Tat, Tat subdomains or mutant Tat protein antigens and detecting at least one antibody characteristic for HIV-1 or a portion thereof or characteristic for HIV-1 infection or an AIDS-associated malignancy.

9. A method of detecting an antibody characteristic for HIV-1 or a portion thereof in a biological sample according to claim 8, the method comprising detecting at least one anti-Tat antibody specificity in a human subject.

10. A method of detecting an antibody characteristic for an AIDS-associated malignancy in a biological sample, the method comprising detecting at least one anti-Tat antibody specificity in a human subject.

11. A pharmaceutical composition for passive immunization of a human subject against HIV-1 infection, the composition comprising at least one monoclonal anti-tat antibody directed against an antigen on the HIV-1 Tat protein.

12. The pharmaceutical composition according to claim 11, wherein the monoclonal antibody is a humanized antibody.

13. The pharmaceutical composition according to claim 11, wherein the monoclonal antibody is a fully human antibody.

14. A method for inducing therapeutic immunity against HIV-1 infection, the method comprising administering at least one antigen characteristic of HIV-1 Tat, Tat subdomain or Tat mutant protein to a human subject.
